(19) United States
(12) Patent Application Publication
(10) Pub. No.: US 2005/0032097 A1
(43) Pub. Date: Feb. 10, 2005

(54) Method for processing samples containing sperm and non-sperm cells for subsequent analysis of the sperm DNA

(76) Inventor: Alex M. Garvin, Durmenach (FR)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/870,639

(22) Filed: Jun. 17, 2004

Related U.S. Application Data
(60) Provisional application No. 60/478,817, filed on Jun. 17, 2003.

(51) Int. Cl. C12Q 1/68; C12N 1/08

(52) U.S. Cl. 435/6; 435/270

(57) ABSTRACT

In various aspects, the present invention provides novel and effective methods and kits for the isolation of sperm and sperm DNA from samples having at least one other cell type, or having the DNA of one other cell type. More specifically, a process for isolating sperm DNA from a mixture of sperm and non-sperm cells by filtration is provided. DNA from non-sperm cells is solubilized by selective lysis, and the intact sperm are retained on a filter, washed, and then treated in situ with a reducing agent to solubilize the sperm DNA. Significantly, centrifugation steps are not required, the process can be easily automated, and can be performed on many samples in parallel. The novel methods and kits are based on filtering selectively solubilized samples through filters that retain sperm by virtue of having uniform pore diameters that: are smaller than sperm, large enough to allow passage of cell debris and solubilized DNA; and are stable to pressure. The inventive methods and kits have broad utility, particularly in the forensic art.

### CROSS-REFERENCE TO RELATED APPLICATIONS

This application claims the benefit of priority, under U.S.C. § 119(e) to U.S. Provisional Application 60/478,817 to Alex M. Garvin, entitled “SEPARATING SPERM FROM SOLUBLE DNA BY FILTRATION,” filed 17 Jun. 2003, the subject matter of which is incorporated in its entirety by reference herein.

### FIELD OF THE INVENTION

The present invention, in various aspects, relates to cell separation and nucleic acid isolation, and more particularly to novel high-throughput methods and kits for separation of sperm and sperm DNA from non-sperm cells and non-sperm cell solubilized DNA. The inventive methods have broad utility, particularly in forensic analyses.

### BACKGROUND

Police departments in the United States currently have a huge backlog of as many as 500,000 unprocessed swabs taken from rape victims. Sperm are normally obtained from a rape victim by rubbing a swab against a mucous membrane, resulting in large numbers of the victim's epithelial cells being collected with the rapist's sperm.

The standard method for purifying sperm from swabs is to first resuspend all cells from the swab and to selectively digest the victim's epithelial cells with a solution containing Proteinase K and SDS. The intact sperm are separated from the solubilized, contaminating DNA by centrifugation, careful removal of supernatant, and extensive washing of the sperm pellet (see e.g., Giusti et al., J Forensic Sci., 31:409-417, 1986; Gill et al. Nature 318:577-579, 1985; Wiegand et al., Int J Legal Med, 104:359-360, 1992; and Yoshida et al., Forensic Sci Int., 72:25-33, 1995). Unfortunately, the processes of centrifugation and careful removal of supernatant are difficult to automate.

For example, Peter Gill et al. (supra) describe a process for isolating sperm DNA from vaginal swabs taken from sexual assault victims. These swabs contain sperm and also a large excess of the victim's epithelial cells. The epithelial cells and the DNA contained in these cells is removed by preferential lysis (i.e., by incubation of the cell mixture in a buffer solution containing SDS (sodium dodecyl sulfate), and proteinase K. Sperm nuclei are impervious to this treatment because they have disulfide bond cross-linked thiol-rich proteins, while other cell types are digested and the corresponding DNA is solubilized. After preferential lysis, the samples are centrifuged to separate the sperm nuclei from the victim's solubilized DNA. The supernatant containing the victim's DNA is removed and the sperm pellet is washed repeatedly. The sperm nuclei are subsequently lysed by treatment with a buffer solution containing SDS, proteinase K and DTT (dithiothreitol).

P. Wiegand et al. (supra) attempt to improve the method of Gill et al. for samples having low sperm counts by using mild lysis conditions and by avoiding the washing steps.

Chen et al. (J Forensic Science 43:114-118, 1998) propose to separate the sperm from the epithelial cells before preferential lysis by gravitational or mild vacuum filtration through a 5-10 μm nylon mesh membrane which is supposed to retain the epithelial cells while the sperm pass through. DNA is then prepared from the sperm collected in the filtrate.

Garvin (PCT/US01/01835, incorporated by reference herein in its entirety) proposes a similar approach to that taken by Chen, et al, but instead of using a nylon mesh filter, a 5 micron polycarbonate track-etch filter is used to separate intact epithelial cells from sperm. Track-etch filters have pores that are stable under pressure (unlike the nylon weave filters used by Chen, et al) and can be subjected to strong vacuum or centrifugal forces without having the pores increase in size.

A number of attempts have been made to circumvent the selective lysis process. For example, Y chromosome polymorphic markers can be amplified from unfractionated swab DNA (Sibille, et al., Forensic Sci Int. 125:212-216, 2002). However the data provided cannot be used to probe the autosomal STR profiles in the FBI CODIS database, it won't work when the rape victim is male, and males of the same paternal lineage usually have identical Y chromosome STR patterns.

Another approach towards avoiding selective lysis is to physically separate sperm from intact epithelial cells. This has been done by flow cytometry (Schoell et al., Obstet Gynecol. 94:623-627, 1999), however this technique is inherently slow due to the need to analyze and sort one cell at a time and is unlikely to be applied to casework.

Attempts have also been made to use anti-sperm antibody coated magnetic beads (Eisenberg, A. J. “Development of a Spermatozoa Capture System for the Differential Extraction of Sexual Assault Evidence”; paper presented at: Profiling PCR and Beyond Conference, 2002; Washington, D.C.). Epitope stability, however, is a likely problem with this approach when applied to casework, because detergents such as Sarkosyl or SDS are required to efficiently elute sperm from the swabs and these detergents destroy most of the epitopes recognized by the anti-sperm antibodies.

Magnetic beads have been successfully used for many cell separation applications (Haukanes & Kvam, Biotechnology (N Y). 11:60-63, 1993), but it remains to be seen if they can be used to separate human cells that have been dried onto an adsorbent substrate and then resuspended.

Sperm can also be physically separated from the much larger intact epithelial cells by size using a 10 micron nylon weave filter (Chen et al., J Forensic Sci. 43:114-118, 1998). Unfortunately the pores of these filters will expand under pressure requiring that only gravity be used as the driving force to minimize the unwanted passage of epithelial cells. In the absence of a strong driving force, capillary action on the filter surface competes with gravity flow through the filter and results in a large retention volume and difficulties with sample handling (present applicant's observation). Furthermore, DNA from epithelial cells lysed by the harsh detergent required for efficient cell re-suspension will pass through the filter with intact sperm.

At the time the Rape Kit DNA Analysis Backlog Elimination Act (Act HR3961, March 2002, was approved by the US Senate, police departments in the United States had as many as 500,000 unprocessed swabs taken from rape victims. This sexual assault backlog problem is due in part to the labor intensiveness and insufficient reliability of the known methods for isolating sperm DNA from casework samples, including the above described methods.

Significantly, at the present time, the rate-limiting step in processing sexual assault cases is that required to separate digested epithelial cells from intact sperm.

Therefore, there is a pronounced need in the art for novel, fast and reliable high-throughput methods for the selective isolation of sperm and sperm DNA from complex samples having sperm along with other cells types, and/or with solubilized DNA from other cell types.

### SUMMARY OF THE INVENTION

In various aspects, the present invention provides novel and effective methods and kits for the isolation of sperm and sperm DNA from samples having at least one other cell type, or the DNA of one other cell type, in addition to sperm. The novel methods and kits are based on filtering selectively solubilized samples through filters that retain sperm by virtue of having uniform pore diameters that: are smaller than sperm, large enough to allow passage of cell debris and solubilized DNA; and are stable to pressure. The inventive methods and kits have broad utility, particularly in the forensic art.

Preferred aspects provide novel methods for isolating sperm DNA, comprising: solubilizing the non-sperm DNA in a sample to provide a selectively solubilized sample; filtering the selectively solubilized sample through a filter that retains sperm, but not solubilized non-sperm DNA; washing the filter; and solubilizing sperm DNA by contacting the retained sperm with a reducing agent.

Additional aspects provide novel methods for isolating sperm, comprising: solubilizing the non-sperm DNA in a sample to provide a selectively solubilized sample; filtering the selectively solubilized sample through a filter that retains sperm, but not solubilized non-sperm DNA; and washing the filter, and optionally recovering the sperm from the filter.

A further preferred embodiment provides a method for separating sperm from non-sperm cell solubilized DNA, comprising: obtaining a sample having sperm and solubilized non-sperm cell DNA; and filtering the sample through a filter, which, based on pore size, retains, or substantially retains sperm, but not the non-sperm cell solubilized DNA.

Additional preferred aspects provide an identification method, comprising: obtaining sperm DNA according to the above described methods; and determining an identity for the sperm donor, based, at least in part, on the isolated sperm DNA. Preferably, the identity determined is that of a perpetrator of a sexual assault.

Further preferred aspects provide kits for isolation of sperm or sperm DNA, comprising a filter, which, based on pore size, retains, or substantially retains sperm, but not solubilized non-sperm DNA.

Preferably, in these above-identified preferred embodiments, the mean diameter of the filter pore size is uniformly at about 2 μm, and is stable to pressure. Preferably, the filter is a track-etch or laser etch filter. Preferably, the filter is a track-etch filter having a mean pore diameter of about 2 μm.

The inventive methods are particularly applicable in the forensic setting for high throughput processing of samples, such as swabs from sexual assault victims, to obtain a sperm DNA profile, wherein such swabs contain in addition to a relatively small amount of sperm a much larger amount of the victim's epithelial cells and possibly blood cells.

Therefore, it is an object of the invention to create methods and kits for processing samples containing a mixture of sperm and other cells (in particular epithelial cells or blood cells) with the aim of analyzing the sperm DNA contained in the sample. Preferred methods are fast, suitable for automation, and provide reliable results even where the amount of sperm present in a test sample is much smaller than the amount of non-sperm cells.

### BRIEF DESCRIPTION OF THE DRAWINGS

FIGS. 1A and 1B show photomicrographs of exemplary size exclusion filters. Filters having the characteristics of the filter shown in FIG. 1B (e.g., a 2 μm ISOPORE™ track-etch filter) are particularly preferred for practicing the methods of the present invention.

FIG. 2 shows a preferred exemplary embodiment of the inventive method for separating, starting with a sample comprising sperm and at least one other cell type, sperm DNA from other cells and solubilized DNA.

FIG. 3 shows standard STR (short tandem repeat) analysis profiles of sperm DNA fractions purified according to an exemplary embodiment of the present invention. The sperm DNA fractions were isolated according to the present invention from a series of vaginal swab cuttings containing, in each case, about 1.3 million epithelial cells, and either: a) 0 sperm; b) 10,000 sperm; c) 20,000 sperm; and d) 50,000 sperm. Profile e) represents a sperm-only control STR profile of a DNA fraction isolated from a swab cutting having only 50,000 sperm, and no epithelial cells. Art-recognized GS500 size standards at 139, 150, 160, and 200 bp are indicated.

### DETAILED DESCRIPTION OF THE INVENTION

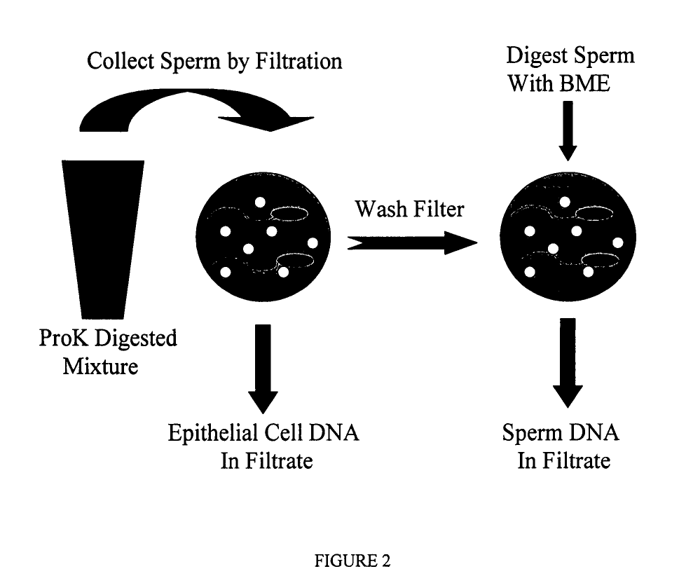

Preferred aspects of the present invention are described with reference to FIG. 2. FIG. 2 shows a preferred exemplary embodiment of the inventive method for separating, starting with a sample comprising sperm and at least one other cell type (or the DNA thereof), sperm DNA from non-sperm cell types and/or from non-sperm cell solubilized DNA.

Overview of preferred method embodiment. In preferred embodiments, separation of sperm and/or sperm DNA from non-sperm cell types and/or from non-sperm cell solubilized DNA is accomplished by: obtaining a sample comprising sperm and at least one other cell type and/or solubilized DNA from at least one other cell type; selectively solubilizing the non-sperm cell DNA (e.g., by selectively lysing the non-sperm cells) in the sample to provide a selectively solubilized sample; filtering the selectively solubilized sample using a filter that, based on pore size, retains sperm, but not solubilized non-sperm DNA; washing the filter to remove or substantially remove any residual solubilized non-sperm DNA; solubilizing the sperm DNA (e.g., in situ on the filter); and collecting the solubilized sperm DNA (e.g., by vacuum filtration through the filter). Preferably, the filter does not clog with DNA or cell debris. A pre-filter is optionally used where clogging of the filter is problematic.

Obtaining a sample containing sperm and at least one of: non-sperm cells having non-sperm cell DNA; or non-sperm cell DNA. A sample comprising sperm, along with at least one other cell type and/or DNA from at least one other cell type, is obtained. For example, such a sample is obtained by rubbing a swab against a tissue (e.g., mucous membrane) of a victim of a sexual assault, thereby providing a swab comprising sperm of a rapist, and epithelial cells from a victim. Alternatively, the sample comprises essentially any mixture of sperm and another somatic cell type (e.g., blood cells, lymphoid cells, etc.). Preferably, the other cell type is a cell type that is susceptible to lysis to release the respective DNA. Preferably, the cell type is susceptible to lysis by treatment with a solution comprising SDS (sodium dodecyl sulfate), and proteinase K. Alternatively, the sample comprises sperm and DNA from non-sperm cells, and the non-sperm DNA is further solubilzed.

Solubilization of non-sperm cell DNA in the sample. In preferred aspects, a sample, comprising sperm, along with at least one other cell type and/or solubilized DNA from at least one other cell type, is placed in a container (e.g., in a well of a 96 deep-well microtiter plate), and the non-sperm cell DNA in the sample is selectively solubilized.

Where the sample comprises sperm and non-sperm cells, solubilization of non-sperm cell DNA is by selective lysis of the non-sperm cells. Any method that selectively lyses non-sperm cells, while leaving the sperm in-tact, or substantially in-tact, is sufficient. Preferably, a combination of SDS and proteinase K is added to the sample to affect selective lysis of non-sperm cells. Preferably, the sample is buffered, SDS is added to about 0.5% (w/v), and proteinase K is added to a concentration of about 2 mg/ml. Alternatively, other standard lysis buffers are known in the art, and one of ordinary skill in the art will be able, without undue effort, to readily determine an optimal combination of, for example SDS and proteinase K to optimally affect selective lysis of non-sperm cells in the sample. Preferably, the sample is first incubated with buffered SDS prior to addition of proteinase K. Alternatively, SDS and proteinase K are added simultaneously or essentially so. Alternatively the proteinase K is added prior to the SDS. Preferably, there is a period of concurrent incubation with SDS and proteinase K (e.g., 30 minutes at 56° C.) sufficient to affect selective lysis of non-sperm cells. Other combinations of selective lysis reagents, and orders of addition are encompassed within the present invention, and optimal combinations and orders are readily determinable by one of ordinary skill in the relevant art.

Where the sample comprises sperm along with non-sperm cell DNA (in addition to, or instead of non-sperm cells), solubilization of the non-sperm cell DNA is affected by the solution or buffered solution used to make the sample (e.g., a buffered solution is added to a swab or swab cutting). Alternatively, additional components (e.g., SDS, proteinase K) are added to an existing liquied sample to further affect solubilizatino by, for example, de-proteinizing any non-sperm chromatin or protein-omplexed non-sperm DNA.

In essence, according to the present invention, conditions to affect selective lysis of non-sperm cells, and/or solubilize non-sperm cell DNA, of non-sperm cells by preferential lysis is carried out using an art-recognized method (e.g., a buffered solution comprising proteinase K and SDS). Preferably, a buffered sample, comprising about 2 mg/ml of proteinase K and about 0.5% SDS is incubated at about 56° C. for about 30 minutes or more to affect solubilization of non-sperm cell DNA.

Filtering the selectively solubilized sample through a filter that, based on pore size, retains or substantially retains sperm, but not the solubilized non-sperm DNA. In preferred aspects, as illustrated by the left filter representation of FIG. 2, separating sperm from non-sperm solublized DNA in the sample (e.g., “ProK digested Mixture”) is affected by filtration, based on filter pore size. Preferably, filtration is pressure based. Preferably filtration is by imposing a differential pressure on opposite sides of a filter. Preferably, the filtration is centrifugation based or vacuum based. Preferably filtration is vacuum based.

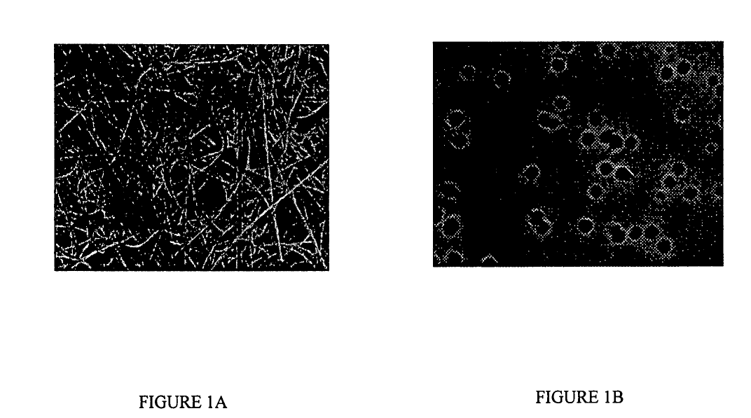

Intact sperm are about 5 μm in diameter while the particulate matter from digested cells (e.g., epithelial cell) has predominately submicron dimensions. Sperm are therefore selectively trapped on a filter having an intermediate pore size of about 2 μm. Where there is a large excess of non-sperm cells, however, the filter must be very selective with minimal trapping of the digested material and solubilized DNA. Most prior art filters have ill-defined pores that consist of a tortuous path through randomly distributed fibers (FIG. 1A), and a filter of this type with a nominal pore size of 2 μm has many pores significantly smaller than 2 μm that trap much non-sperm cell solubilized DNA as well as sperm. Therefore, according to the preferred aspects of the present invention, track-etched (e.g., ISOPORE™ track-etch) or laser-eteched filters, which have precisely defined pores, are ideally suited for the instant inventive applications. The advantages of track-etch filters for the instant purposes are the high accuracy of the dimensions of the openings, and the fact that these dimensions are stable under pressure (e.g., centrifugation, vacuum, etc.). Such track edge filters are available under the trade name ISOPORE from Millipore Corporation. Track-etch filters are made, for example, by subjecting a polymer membrane (e.g., a polycarbonate membrane) to high-energy radiation and then etching away the impact points (e.g., in an acid bath) to produce circular pores of well defined size that go straight through the membrane (FIG. 1B). FIG. 1A shows a photomicrograph of a typical polypropylene depth filter. FIG. 1B shows a photomicrograph of a micron ISOPORE™ track-etch filter.

Therefore, in preferred aspects, the filter has a mean pore diameter that is stable, or substantially stable under pressure (e.g., under centrifugation, or vacuum conditions). Preferably, the mean pore diameter is greater than about 1 μm, and less than about 3 or 4 μm. Preferably the mean pore diameter is about 2 μm. Preferably, the filter has substantially uniform pores of a defined diameter. Preferably, the filter is a filter produced by track etching or laser etching. Preferably the filter is a track-etched filter. Preferably, filtering comprises use of a track-etched filter having a mean pore diameter of about 2 μm. Preferably, at least 50% of the pores differ in diameter by no more than 40% from the mean pore diameter.

Preferably, filtering additionally comprises use of a pre-filter to preclude clogging of the filter by debris present in the sample (e.g., cotton swab material, etc.). Preferably, the pre-filter is a nylon mesh filter having a mesh of about 11 μm. For example, clogging is prevented by placing an 11 micron nylon net pre-filter directly above a 2 μm track-etch filter, the filters are placed in a 96-well holder, and sperm are separated from digested non-sperm cells (e.g., epithelial cells) by vacuum-driven filtration.

Preferably, filtering the selectively solubilized sample through the filter(s) is centrifugation-based (e.g., using a modest centrifugal force of about 50-times gravity), or vacuum-based. Accordingly, the inventive method is highly suitable for high-throughput applications; that is, preferred embodiments are carried out in an automated manner using, for example, 96-well microtiter plates (96-well filter plates and installations designed for processing such plates are widely known and recognized in the art).

Washing the filter to remove, or substantially remove any residual solubilized non-sperm DNA therefrom. Preferably, the filter or filter stack on which the sperm cells are retained is washed to remove as much of the solubilized DNA as possible. The wash solution can be any solution suitable to wash solubilized DNA through the filter, while not disrupting the retained sperm. Preferably, the washing is done using 1× BackGround Quencher™ (MRC, Inc.) in ATL buffer (see below) followed by distilled water.

Preferably, non-specific binding of non-sperm solubilized DNA to the filters is reduced by blocking the filter(s) to preclude DNA binding by exposing the filter(s) to suitable blocking reagents. Many such agents are known in the arts, including, but are not limited to BackGround Quencher™ (MRC, Inc.),and including those art-recognized reagents used to block non-specific DNA binding to Southern blotting filters.

Solubilizing sperm DNA by contacting the retained sperm with a reducing agent suitable to disrupt sperm. In particular aspects, retained sperm are isolated by recovering them from the filter. This is, for example, done by gentile agitation in a suitable buffer for a sufficient time, and/or by reverse filtration.

Alternatively, and preferably, the sperm are digested in-situ on the filter using high concentrations of an art-recognized reducing agent. Solubilizing the DNA from the retained sperm is carried out using art-recognized agents suitable to disrupt the retained sperm. Preferably, the reducing agent comprises an agent selected from the group consisting of β-mercaptoethanol (BME), dithiothreitol (DTT), reduced glutathione (GSH), and combinations thereof. Preferably, the reducing agent comprises β-mercaptoethano, DTT, or combinations thereof. Other useful reagents are well known in the art and, for example, can be found in Sambrook, Fritsch and Maniatis, Molecular Cloning: A Laboratory Manual, CSH Press, 2nd edition, 1989: Isolation of genomic DNA from mammalian cells, Protocol I, p. 9.16-9.19.

Significantly, however, according to the present invention, substantially higher levels of reducing agents are needed to affect fast and efficient digestion of sperm on a filter. For example, the typical art-recognized concentrations of BME used to digest soluble sperm is 1%. Likewise, for example, forensics labs typically use about 20-40 mM DTT to lyse sperm in solution. However, 20-40 mM DTT is not enough to effectively lyse sperm heads immobilized on a filter.

Preferably, according to the present invention, 5 to 10% BME is used at room temperature for 5 minutes to affect retained-sperm sperm DNA solubilization. Preferably, about 10% BME is used at room temperature for about 5 minutes. Preferably, the reducing agent is DTT. Preferably, the concentration of DTT required to disrupt sperm retained on the filter is about 0.5 M to about 2 M. Preferably, the concentration of DTT is about 2M.

Isolating the solubilized sperm DNA. The step of collecting the soluble DNA is carried out by washing the filter or filter stack. For example, when about 10% BME, or about 2 M DTT is used at room temperature for 5 minutes, the retained-sperm sperm DNA readily goes into solution, and can be passed through the filter and collected.

In the preferred steps described above, the force required to move solutions through the inventive filters (e.g., 2 μm track-etch filter) is modest. For example, the inventive filtration can be accomplished using centrifugal force equal to about 50× gravity, or by vacuum. The use of a vacuum has the advantage that no centrifugation steps are required, thus the entire process of separating sperm DNA from non-sperm cells and non-sperm cell DNA can be accomplished by pipetting, temperature controlled incubations, and vacuum-driven filtration. These advantages enable implementation of high-throughput methods and assays.

Therefore, in view of the above disclosure, a preferred aspect the invention provides a method for isolating sperm DNA, comprising: obtaining a sample containing sperm, and at least one of non-sperm cells having non-sperm cell DNA or non-sperm cell DNA; solubilizing the non-sperm DNA to provide a selectively solubilized sample; filtering the selectively solubilized sample through a filter, which, based on pore size, retains, or substantially retains sperm, but not the solubilized non-sperm DNA; washing the filter to remove, or substantially remove any residual solubilized non-sperm DNA therefrom; solubilizing sperm DNA by contacting the retained sperm with a reducing agent suitable to disrupt sperm; and isolating the solubilized sperm DNA.

An additional preferred embodiment provides a method for isolating sperm, comprising: obtaining a sample containing sperm, and at least one of non-sperm cells having non-sperm cell DNA or non-sperm cell DNA; solubilizing the non-sperm DNA to provide a selectively solubilized sample; filtering the selectively solubilized sample through a filter, which, based on pore size, retains, or substantially retains sperm, but not the solubilized non-sperm DNA; and washing the filter to remove, or substantially remove any residual solubilized non-sperm DNA therefrom.

A further preferred embodiment provides a method for separating sperm from non-sperm cell solubilized DNA, comprising: obtaining a sample having sperm and solubilized non-sperm cell DNA; and filtering the sample through a filter, which, based on pore size, retains, or substantially retains sperm, but not the non-sperm cell solubilized DNA.

Yet additional preferred embodiments provide An identification method, comprising: obtaining sperm DNA according to the method of any one of claims 1-18; and determining an identity for the sperm donor, based, at least in part, on the isolated sperm DNA. Preferably, the identity determined is that of a perpetrator of a sexual assault.

Yet further preferred embodiments provide a kit for isolation of sperm or sperm DNA, comprising a filter, which, based on pore size, retains, or substantially retains sperm, but not solubilized non-sperm DNA.

Preferably, in these above-identified preferred embodiments, the mean diameter of the pore size is uniformly at about 2 μm, and is stable to pressure. Preferably, the filter is a track-etch or laser etch filter. Preferably, the filter is a track-etch filter having a mean pore diameter of about 2 μm.

The present invention is further illustrated by reference to the EXAMPLES below. However, it should be noted that these EXAMPLES, like the embodiments described above, are illustrative and are not to be construed as restricting the enabled scope of the invention in any way. The references cited herein above are all incorporated by reference herein.

### EXAMPLE I

### Sperm DNA from Vaginal Swab Samples was Isolated According to a Preferred Filtration Embodiment of the Present Invention and then Analyzed

Sperm-free vaginal swabs and semen were taken from healthy volunteers and the cells were counted using a hemocytometer. Vaginal swab cuttings (one half of a swab) each having approximately 1.3 million epithelial cells were spiked with 2 μl, 4 μl and 10 μl of a 10% semen solution having 5000 sperm per μl (10,000, 20,000 and 50,000 sperm per swab cutting). The spiked cuttings were left at room temperature for two weeks. The cuttings were then placed in a 96 deep-well microtiter plate with 600 μl of Qiagen™ ATL buffer (which contains SDS) and shaken for 30 min at room temperature at 500 rpm on a rotating shaker. The cotton material from the swab was removed with tweezers and pressed against the side of the well to reduce liquid loss, leaving about 500 μl of ATL buffer in the well. 25 μl of a 500 μg/ml proteinase K solution was added, mixed by pipetting, and incubated at 56° C. for 1 h.

Sperm were collected on a filter stack consisting of a 10 micron nylon net (Millipore) placed on top of a 2 micron ISOPORE™ track-etch filter (Millipore). The filters were treated with 10× BackGround Quencher (MRC, Inc.) for 30 min at room temperature to reduce the binding of free DNA. Seven millimeter filter disks were made using a hammer and a disk punch and placed in the wells of a Qiafilter™ 96-plate whose provided filters had been removed. Vacuum, at 200 absolute Torr, was applied using a QIAvac™ 96-manifold (Qiagen), and the epithelial cell DNA was collected in the filtrate. Filters were washed with 500 μl of 1× BackGround Quencher in ATL buffer followed by 3 ml of distilled water. Sperm DNA was then solubilized by treating the filters with 50 μl of 10% solution of BME in ATL buffer for 30 min at room temperature. The filters were then washed with 150 μl ATL and 200 μl AL buffer (Qiagen) and the filtrate containing sperm DNA was collected in micro tubes in the manifold.

DNA was purified from both the epithelial and sperm fractions using Qiamp™ mini-columns with a final elution volume of 50 μl (Qiagen). Quantitation was done by ethidium bromide staining on a 1% agarose gel. 5 μl of DNA was used as template for 30 cycles of PCR using primers for locus D21S1435, a locus for which the sperm and epithelial cell DNA samples of this study have no alleles in common. The 5′ primer was labeled with the Hex dye. Fragments were analysed on an ABI 310 single-capillary automated sequencer using GS500 size standards and peak areas were determined using Genescan software (Applied Biosystems).

The epithelial cell DNA generates a 170 base-pair amplification product, while the sperm DNA generates 186 and 190 base-pair products. With 10,000 sperm on the swab cutting, the signal of the sperm fraction is predominantly epithelial, but with 20,000 sperm the combined sperm peaks represent 61% of the total signal. With 50,000 sperm the sperm signal is dominant with 86% of the total signal. The initial sample spiked with 50,000 sperm contained 7.8 μg of epithelial DNA and about 150 ng (1.9%) of sperm DNA. The processed sample with 86% of sperm specific signal therefore constitutes a 45-fold enrichment.

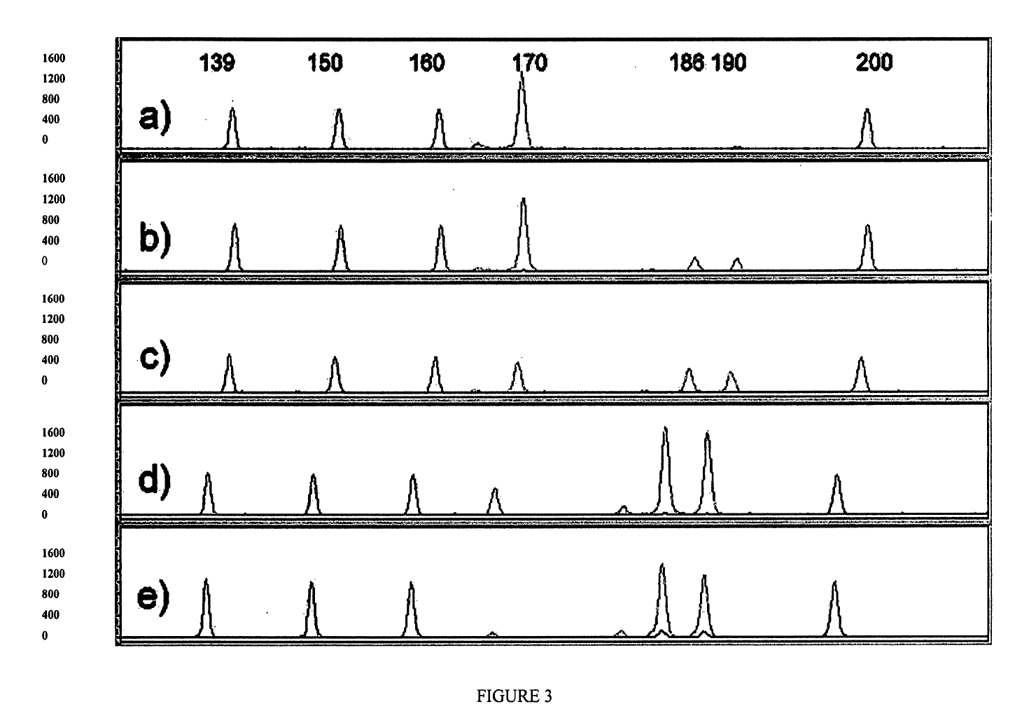

Specifically, FIG. 3 shows standard STR (short tandem repeat) analysis profiles of sperm DNA fractions purified according to an exemplary embodiment of the present invention. The sperm DNA fractions were isolated according to the present invention from a series of vaginal swab cuttings containing, in each case, about 1.3 million epithelial cells, and either: a) 0 sperm; b) 10,000 sperm; c) 20,000 sperm; and d) 50,000 sperm. Profile e) represents a sperm-only control STR profile of a DNA fraction isolated from a swab cutting having only 50,000 sperm, and no epithelial cells. Art-recognized GS500 size standards at 139, 150, 160, and 200 bp are indicated.

1. A method for isolating sperm DNA, comprising:
obtaining a sample containing sperm and at least one of: non-sperm cells having non-sperm cell DNA; or non-sperm cell DNA;
solubilizing the non-sperm DNA to provide a selectively solubilized sample;
filtering the selectively solubilized sample through a filter that, based on pore size, retains, or substantially retains sperm, but not the solubilized non-sperm DNA;
washing the filter to remove, or substantially remove any residual solubilized non-sperm DNA therefrom;
solubilizing sperm DNA by contacting the retained sperm with a reducing agent suitable to disrupt sperm; and
isolating the solubilized sperm DNA.

2. A method for isolating sperm, comprising:
obtaining a sample containing sperm and at least one of: non-sperm cells having non-sperm cell DNA; or non-sperm cell DNA;
solubilizing the non-sperm DNA to provide a selectively solubilized sample;
filtering the selectively solubilized sample through a filter that, based on pore size, retains, or substantially retains sperm, but not the solubilized non-sperm DNA; and
washing the filter to remove, or substantially remove any residual solubilized non-sperm DNA therefrom.

3. The method according to claim 2, further comprising recovering the sperm from the filter.

4. The method according to any one of claims 1 through 3, wherein filtering comprises use of a filter having a mean pore diameter that is stable, or substantially stable under pressure.

5. The method according to claim 4, wherein the mean pore diameter is about 2 μm.

6. The method according to any one of claims 1 through 3, wherein filtering comprises use of a filter selected from the group consisting of a track-etched filter, a laser-etched filter, and combinations thereof.

7. The method according to any one of claims 1 through 3, wherein filtering comprises use of a track-etched filter.

8. The method according to any one of claims 1 through 3, wherein filtering comprises use of a track-etched filter having a mean pore diameter of about 2 μm.

9. The method according to claim 8, wherein at least 50% of the pores differ in diameter by no more than 40% from the mean pore diameter.

10. The method according to any one of claims 1 through 3, wherein filtering additionally comprises use of a pre-filter.

11. The method according to claim 10, wherein the pre-filter is a nylon mesh filter having a mesh of about 11 μm.

12. The method according to any one of claims 1 through 3, wherein filtering comprises vacuum filtration.

13. The method according to any one of claims 1 through 3, wherein filtering comprises centrifugation-based filtration.

14. The method according to claim 10, wherein at least one of the filter or prefilter is pre-treated with a blocking agent to reduce binding of solubilized DNA thereto.

15. The method according to any one of claims 1 through 3, wherein the reducing agent comprises an agent selected from the group consisting of β-mercaptoethanol, dithiothreitol (DTT), reduced glutathione (GSH), and combinations thereof.

16. The method according to claim 15, wherein the reducing agent comprises β-mercaptoethanol.

17. The method according to claim 1, wherein isolating the solubilized sperm DNA is by vacuum filtration through the filter into a receiving container.

18. The method according to any one of claims 1 through 3, wherein the sample is obtained from a victim of a sexual assault.

19. A method for separating sperm from non-sperm cell solubilized DNA, comprising:
obtaining a sample having sperm and solubilized non-sperm cell DNA; and
filtering the sample through a filter that, based on pore size, retains, or substantially retains sperm, but not the non-sperm cell solubilized DNA.

20. The method according to claim 19, wherein filtering comprises use of a filter having a mean pore diameter that is stable, or substantially stable under pressure.

21. The method according to claim 20, wherein the mean pore diameter is about 2 μm.

22. The method according to any one of claims 19 through 21, wherein filtering comprises use of use of a filter selected from the group consisting of a track-etched filter, a laser-etched filter, and combinations thereof.

23. The method according to any one of claims 19 through 21, wherein filtering comprises use of a track-etched filter.

24. The method according to any one of claims 19 through 21, wherein filtering comprises use of a track-etched filter having a mean pore diameter of about 2 μm.

25. The method according to claim 24, wherein at least 50% of the pores differ in diameter by no more than 40% from the mean pore diameter.

26. The method according to any one of claims 19 through 21, wherein filtering additionally comprises use of a pre-filter.

27. The method according to claim 26, wherein the pre-filter is a nylon mesh filter having a mesh of about 11 μm.

28. The method according to any one of claims 19 through 21, wherein filtering comprises vacuum filtration.

29. The method according to any one of claims 19 through 21, wherein filtering comprises centrifugation-based filtration.

30. The method according to claim 26, wherein at least one of the filter or prefilter is pre-treated with a blocking agent to reduce binding of solubilized DNA thereto.

31. A kit for isolation of sperm or sperm DNA, comprising a filter that, based on pore size, retains, or substantially retains sperm, but not solubilized non-sperm DNA.

32. The kit according to claim 31, wherein the filter has a mean pore diameter that is stable, or substantially stable under pressure.

33. The kit according to claim 32, wherein the mean pore diameter is about 2 μm.

34. The kit according to any one of claims 31 through 33, wherein the filter is selected from the group consisting of a track-etched filter, a laser-etched filter, and combinations thereof.

35. The kit according to any one of claims 31 through 33, wherein the filter is a track-etched filter.

36. The kit according to any one of claims 31 through 33, wherein the filter is a track-etched filter having a mean pore diameter of about 2 μm.

37. The kit according to claim 36, wherein at least 50% of the pores differ in diameter by no more than 40% from the mean pore diameter.

38. The kit according to any one of claims 31 through 33, further comprising a pre-filter.

39. The kit according to claim 38, wherein the pre-filter is a nylon mesh filter having a mesh of about 11 μm.

40. The kit according to any one of claims 31 through 33, wherein the filter is suitable for vacuum-based filtration.

41. The kit according to any one of claims 31 through 33, wherein the filter is suitable for centrifugation-based filtration.

42. The kit according to any one of claims 31 through 33, comprising an array of such filters.

43. An identification method, comprising:
obtaining sperm DNA according to the method of any one of claims 1-18; and
determining an identity for the sperm donor, based, at least in part, on the isolated sperm DNA.

44. The method of claim 43, wherein the identity determined is that of a perpetrator of a sexual assault.
